(19) United States
(12) Patent Application Publication
(10) Pub. No.: US 2023/0399619 A1
(43) Pub. Date: Dec. 14, 2023

(54) DEVICE AND METHOD FOR MAKING CELL SHEETS

(71) Applicant: CaroMeats Inc., Kitchener (CA)

(72) Inventor: Alireza Shahin-Shamsabadi, Kitchener (CA)

(73) Assignee: CaroMeats Inc., Kitchener (CA)

(21) Appl. No.: 18/207,165

(22) Filed: Jun. 8, 2023

Related U.S. Application Data
(63) Continuation-in-part of application No. 17/882,693, filed on Aug. 8, 2022, now Pat. No. 11,718,830.
Application No. 17/882,693 is a continuation of application No. 17/838,284, filed on Jun. 13, 2022, now abandoned.
(60) Provisional application No. 63/504,774, filed on May 29, 2023.

(51) Int. Cl. C12N 5/077; B33Y 40/00; B33Y 10/00

(52) U.S. Cl. CPC C12N 5/0658; B33Y 40/00; B33Y 10/00; C12N 2537/00; C12N 2513/00; C12N 2535/10; C12N 2533/30; C12N 2533/70; C12N 2500/12; C12N 2533/20; C12N 2537/10

(57) ABSTRACT

Systems and methods for growing cells or cell sheets are described. The method may use biocompatible or food-safe materials. The method may allow for cell alignment, optionally in selected patterns, which may be produced by casting a membrane on a 3D printed mold. The method may include surface treatment of an elastomeric membrane. The method may allow for the reuse of the membranes by autoclaving and/or a washing step. The method may create multi-layer cell sheets with an extracellular matrix (ECM) created by the cells, which may be detached from the membrane. Optionally, the cells may be separated from the ECM.

### CROSS REFERENCE TO RELATED APPLICATIONS

This application claims the benefit of U.S. Provisional Application Ser. No. 63/504,774, filed May 29, 2023, and is a continuation-in-part of U.S. application Ser. No. 17/882,693, filed Aug. 8, 2022, which is a continuation of U.S. application Ser. No. 17/838,284, filed Jun. 13, 2022. Application Ser. Nos. 63/504,774; 17/882,693; and 17/838,284 are hereby incorporated by reference.

### FIELD OF THE INVENTION

The field of this invention relates to devices and methods for growing cells or making cell sheets. Some examples involve substrate surface chemistry, substrate topography, or methods of processing cell sheets.

### BACKGROUND OF THE EMBODIMENTS

Silicone-based polymer family members, such as PDMS, have favorable features for cell culture related applications, including their transparency and ease of microscopy, non-toxic and inert nature both chemical and biological, as well as their gas permeability. On the other hand, their intrinsic hydrophobicity results in non-specific protein adsorption, conformation and denaturation, as well as inappropriate cell attachment, agglomeration, and detachment (Walsh, 2017).

Multiple methods have been proposed to enhance surface hydrophilicity and improve cell attachment of the silicone-based elastomers, such as PDMS. For example, some groups propose use of high energy plasma radiation, which is a fast method, but the created Si-hydroxyl groups are short lived and migration of molecules from the bulk of the material can neutralize its effect (Fritz, 1995).

Other groups have proposed use of covalent immobilization of extracellular matrix proteins on the surface of the PDMS as an example method. However, this method requires functionalization of the PDMS surface by salinization and addition of linkers, such as 1-ethyl-3-(3-dimethylaminopropyl)carbodiimide/N-hydroxysuccinimide (EDC/NHS), glutaraldehyde, and polymer brushes. These modifications require multi-step and complicated processes involving harsh and often toxic materials which could leave residues and the use of organic solvents that can swell PDMS and damage its surface features (Li, 2018).

As another example, some groups have proposed coating the PDMS with dopamine (Chuah, 2015). However, dopamine monomers are expensive, potentially toxic, and the dark nature of the coating interferes with microscopy. Other proposed methods of improving PDMS cell attachment include grafting with polyethylene glycol or PEG after plasma treatment (Sharma, 2007) and surface modification with Pluronic F68, poly-L-lysine, and fibronectin (Wu, 2009).

Further, some groups have proposed coating PDMS through the formation and deposition of iron(III)-tannic acid networks (Lv, 2020).

Cell sheet engineering was originally introduced in 1990s using cell culture surfaces, tissue culture polystyrene in particular, grafted with N-isopropylacrylamide (PIPAAm). PIPAAm is a temperature sensitive material that allows for detachment of cells and their secreted extracellular matrix (ECM) proteins without using enzymes, such as trypsin, that digest and destroy ECM proteins. This results in an intact cellular construct in the form of a sheet by decreasing the temperature and changing the hydrophilicity of the grafted polymer (Yamada, 1990). Alternatively, other responsive surfaces have been created for this purpose by layer-by-layer deposition of cationic and anionic polyelectrolytes on indium tin oxide (ITO). The trigger for starting the delamination of cell sheets in this case is lowering the pH as these layers become unstable at low pH and will not allow cell attachment (Guillaume-Gentil, 2011).

Another cell sheet formation technique includes growing cells on a feeder layer and detaching them by using another enzyme, dispase, that digests some of the ECM proteins and not cell-cell junctions. Growing cells on an amniotic membrane as a carrier and then using them with the membrane has been proposed as well. Using surfaces sensitive to other external stimuli such as light, electrochemical polarization, ionic solution, and magnetic force has also been used for making cell sheets (Owaki, 2014). A more recent technique that does not rely on characteristics of the surface for making cell sheets uses slight changes in the pH of the environment to cause cells to show contraction and detach from the surface. This method is only applicable to cells that show syncytialization and fusion such as skeletal muscle cells (Shahin-Shamsabadi, 2020). Thus, alternative systems and methods for creating cell sheets are needed.

Example related art includes:

US20210180012A1 describes a method of fabricating curvature-defined (C-D) or shape-defined (S-D) convex and concave gel surfaces for use in cell and tissue culturing and in other surface and interface applications, and provides a method of using C-D or S-D convex and concave surfaces with varying curvatures to direct cell attachment, spreading, and migration.

U.S. Pat. No. 5,776,747A describes a method of derivatizing or adsorbing polyethylene oxide-poly(dimethylsiloxane) copolymer (PEO-PDMS) onto a surface within a bioartificial organ to inhibit cellular attachment.

KR102215710B1 describes a multilayer foam sheet of an acrylonitrile butadiene styrene (ABS) resin, which is a thermoplastic resin having excellent mechanical strength and low-temperature shock resistance, a manufacturing method thereof, and a three-dimensional (3D)-molded product manufacturing method using the sheet.

IN201721015210A describes a bio-inspired 3D-micro/nanofluidic device manufactured by a scalable fabrication process. The device comprises thin micro/nano vascularized membrane matrices made of PDMS or dimethicone.

WO2018058135A1 describes photocurable poly(siloxane) formulations for making stereolithographic 3D-printed PDMS structures, stereolithographic 3D-printing methods for making PDMS structures, and stereolithographic 3D-printed PDMS structures.

CN105504759B describes an ABS composite material used for 3D printing.

CN106046700A describes a method for preparing a 3D printing material from PETG plastic and a vegetable fiber.

### SUMMARY OF THE EMBODIMENTS

The present invention and its embodiments relate to apparatus and methods for creating cell constructs such as cell sheets. In some examples the cell constructs have high ECM content. In some examples, the cell constructs have controlled cell alignment. The ECM is a three-dimensional (3D) network that, depending on the tissue, can consist of extracellular macromolecules and/or minerals, such as collagen, enzymes, glycoproteins and hydroxyapatite, that provide structural and biochemical support to surrounding cells. The cell sheets can be made with any cell type from any species. The technique of the present invention may be simple, easy to perform, have a low-cost, or use biocompatible, non-toxic, food-grade or food-safe materials.

In some examples, the present invention allows for cell alignment, optionally in selected or sophisticated patterns. The patterns of cell alignment are induced by patterns in a membrane, optionally made using molding processes with 3D printed molds. Molds may be prepared with 3D printers and using different types of filament materials. Similar patterns can alternatively be created on a mold, for example using CNC machining or laser engraving, on different materials. It is also possible to make the patterns directly on the membrane, for example using CNC machining or laser engraving, without the need to use a patterned mold. Moreover, the present invention optionally provides for the reuse of membranes by autoclaving and/or washing with an appropriate solvent with defined polarity to induce controlled and limited swelling in the membrane that helps with the release of the absorbed elements, for example washing a PDMS membrane with isopropyl alcohol. Additionally, the present invention may provide multi-layer cell constructs, induced ECM production, or ECM crosslinking using biocompatible or food-grade materials. The method may result in sheets, optionally formed in a short time (usually 518 days), that can be partially or completely removed from a membrane using scraping.

An example embodiment described herein includes a method to create a cell construct such as a cell sheet. The method may include process steps such as: treating an elastomeric membrane with an aqueous solution (e.g., an aqueous solution of tannic acid and/or an aqueous solution of lignin). In some examples, the elastomeric membrane is treated directly with about 10-200 mg/mL of the aqueous solution of tannic acid or about 0.1-5 mg/mL of the solution of lignin at room temperature for a time period of about 6 hours to 4 days. In some examples, the method also includes treating the surface of the treated elastomeric membrane with a solution (e.g., a sodium hydroxide solution in deionized water) or plasma. Elastomeric membranes can be pre-treated with aqueous solution of sodium hydroxide with 0.1-5 molar concentrations for 12 hours up to 5 days before treatment with tannic acid or lignin solutions are performed to enhance their effect. If pre-treated with sodium hydroxide or other techniques, depending on the elastomer, lower concentrations and shorter treatment times of either tannic acid or lignin can potentially have similar effects as their higher concentrations and longer treatment times.

Next, the method includes growing cells on the treated elastomeric membrane and aligning the cells on the treated elastomeric membrane. The aligning of the cells on the treated elastomeric membrane occurs by creating patterns on a surface of the treated elastomeric membrane. In some examples, aligning the cells on the treated elastomeric membrane occurs by utilizing three-dimensional (3D) printing to create a master mold with patterns that can be replicated to the treated elastomeric membrane by casting. In some implementations, the 3D printing comprises Fused Deposition Modeling (FDM) 3D printing. Alternatively, stereolithography (SLA) based 3D printing can be used to create defined patterns similar to the ones created by FDM methods. It should be appreciated that the patterns (e.g., a parallel pattern, a circular pattern, a concentric pattern, or a more complex pattern) are used as topographical signaling cues for the cells to be aligned in certain directions.

Further, the method includes detaching the cell construct (i.e. one or more layers of cells and ECM produced by the cells) from the membrane. In some examples, detaching the cell construct creates a cell sheet, in particular a detached cell sheet. Detaching the cell construct may occur spontaneously as the cell construct grows, or may be initiated by a force such as scraping or shaking, or may be performed by one or more physical actions such as scraping, scratching, shaking or pulling. Detaching the cell construct may occur without applying a stimuli to trigger a change in a responsive surface (e.g. a surface responsive to a change in temperature, an ionic solution, pH, light, magnetic force or electrochemical polarization) attached to the membrane. Detaching the cell construct may occur without inducing contraction of the cells. Detaching the cell construct may occur without applying a chemical agent such as an enzyme.

In some examples, the method additionally includes: inducing the cells to produce a sufficient amount of extracellular matrix (ECM) by treating the cells with an ascorbate or by macromolecular crowding, for example by adding a polyethylene glycol (PEG) or carrageenan to media. The method may also include: determining that the cells are confluent and have produced the sufficient amount of ECM. The method may also include growing additional cells on the initial cell-ECM layer.

In some examples, the method additionally includes: inducing the cells to produce a sufficient amount of extracellular matrix (ECM) by treating the cells with an ascorbate or by macromolecular crowding, for example by adding a polyethylene glycol (PEG) or carrageenan to media. Optionally, the method may include cross-linking the ECM produced by the cells with significantly lower concentrations of tannic acid (less than 0.1 mg/mL) or lignin (less than 10 μg/mL) for much shorter times (5-30 minutes) to create a stable structure and removing the cells and ECM of the cells from the surface of the elastomeric membrane to create the cell sheet.

In some examples, the method may include: determining that the cells are confluent and have produced the sufficient amount of ECM. The method may also include growing additional cells on the initial cell-ECM layer.

Removal of the cells and the ECM of the cells from the surface of the elastomeric membrane can occur by scraping the cells and the ECM of the cells, or a portion thereof, off of the surface of the elastomeric membrane. In some examples, the method may include washing and autoclaving the treated elastomeric membrane to reuse the treated elastomeric membrane to grow new cells and make new cell sheets.

The present invention illustrates the possibility of using natural polyphenols such as tannic acid in its solution format with proper solvents such as deionized water and their effect on the physical or chemical properties (e.g. hydrophilicity) of the elastomeric membranes in order to enhance cell attachment. Elastomeric membranes may include silicone-based materials such as PDMS or other elastomers including but not limited to poly(butylene adipate-co-terephthalate) (PBAT) and urethane rubbers. Similarly, lignin is shown in this application to alter the hydrophilicity of elastomeric membranes and may be applied using solvents such as deionized water and methanol. The effects of these treatments are stable and can withstand autoclaving and washing steps with organic solvents, such as isopropyl, that can cause elastomer swelling. A membrane, for example an elastomeric membrane, made be provided with a hydrophilic surface without adding an exogenous ECM component to the membrane.

In some examples, the steps can also be adapted for use with non-elastomeric membranes for example, hydrophilic polycarbonate, polysulfone, and polyethersulfone.

The specification also describes a polyphenol-coated substrate for growing a cell sheet. The substrate may include an elastomeric membrane and one or more polyphenols attached to the surface of the elastomeric membrane. The polyphenol may include tannic-acid. The substrate may be made without iron. For example, the tannic acid may be un-cross-linked, or not bound to iron. In some examples, the polyphenol is lignin. The elastomeric membrane may comprise a silicone, for example PDMS, or PBAT. The substrate may have a contact angle that is at least 20, 40 or 55 degrees and at most 70, 75, 80, 85 or 90 degrees. The substrate may have a portion having parallel grooves with a spacing in the range of 0.01-500 microns or 5-50 microns.

This specification also describes a method of making a polyphenol coated substrate. The method includes treating an elastomeric membrane with an aqueous solution of a plant-based polyphenol, optionally in the substantial absence of iron, metals and/or added cross-linkers. The aqueous solution may include tannic acid, for example at least 10 mg/ml of tannic acid, at least 25 mg/mL of tannic acid or at least 50 mg/mL of tannic acid, optionally up to 100 mg/mL of tannic acid or up to 200 mg/mL. The aqueous solution may have a pH of 7.5 or less. Alternatively or additionally, the aqueous solution may comprise lignin, for example at least 0.1 mg/mL of lignin, at least 1 mg/mL of lignin or at least 2 mg/mL of lignin, optionally up to 4 mg/mL or up to 6 mg/mL of lignin. The substrate may be treated with a basic solution, for example of NaOH, before treating the elastomeric membrane with the aqueous solution of the plant-based polyphenol. The membrane may be made of a silicone, for example polydimethylsiloxane (PDMS), or PBAT. The membrane may be treated with the aqueous solution for a time period of about 6 hours to 4 days.

This specification also describes a method of making a cell sheet. The method includes creating a patterned membrane; and, growing cells on the patterned membrane in layers wherein the layers include ECM produced by the cells. A layer may be formed by adding cells to 60-90% confluence and incubating the cells to full confluence. Multiple cell layers may attach or fuse together to form a cell sheet. The patterns may be created by casting the membrane on a mold made by three-dimensional (3D) printing, wherein patterns of the mold are replicated on the membrane. The 3D printing may be Fused Deposition Modeling (FDM) 3D printing. The pattern may include grooves, for example in a parallel pattern, a circular pattern, and a concentric pattern. The cells may be induced to produce more extracellular matrix (ECM) components by treating the cells with an ascorbate, or by macromolecular crowding, for example by adding polyethylene glycol (PEG) or carrageenan to media. The ECM may be cross-linked, for example with tannic acid or lignin. The cells and ECM of the cells may be removed from the surface of the membrane as a cell sheet, for example by scraping at least a portion of the layers from the surface of the membrane. The remainder of the layers may be removed from from the membrane, for example, by scraping, pulling, shaking or spontaneous detachment. The call sheet may be rolled to form a cellular fiber. A plurality of cell sheets or cell fibers may be stacked together.

This specification also describes a patterned membrane. The membrane may have a region with parallel grooves with a spacing of 0.01 to 500 microns. The membrane may have multiple sets of groves with different spacings, for example spacings of 5-99 or 5-50 microns, spacings of 100-500 microns and/or spacings 0.01 to 5, 0.01-1 microns, or 0.1-1 microns. The membrane may comprise an elastomer, optionally with a polyphenol coating.

This specification also describes a method of making a patterned membrane. The method includes making a mold by way of fused deposition modeling 3D printing and casting a membrane on the mold. The nozzle size of the 3D printer may be 0.1-0.6 mm. The printing speed may be 1-100 mm/s. The mold may be treated with a solvent vapor before casting the membrane on the mold. A filament may be laid down in parallel lines in at least a portion of the mold. The membrane may be an elastomer.

This specification also describes a method of making a cell sheet with stimulation. The method includes growing a cells in one or more layers on a membrane and mechanically or electrically stimulating the cell layers. The membrane may be stretched in a repeated cycle, for example to provide static stimulation or dynamic stimulation. The method may include applying a fluctuating voltage to the cell layers.

This specification also includes a method of increasing ECM production. The method includes creating a membrane; growing cells on the membrane in layers, and inducing the cells to produce more extracellular matrix (ECM) components and/or crosslinking the ECM. The cells may be treated with an ascorbate. A crowding agent, for example polyethylene glycol (PEG) or carrageenan, may be added to media. The ECM produced by the cells may be crosslinked, for example with tannic acid or lignin.

This specification also describes a method of making ECM. The method includes growing cells in layers to form a cell construct and decellularizing the cell construct. The decellularized cell construct may be solubilized and DNA residues may be removed. The solubilized ECM may be dried, for example freeze dried. An ECM gel may be reconstituted from solubilized or dried ECM. The ECM may be used as a coating or scaffold for growing cells, for example growing human cells.

The apparatus elements and steps described above can be used alone or in any permutation or sub-combinations.

### BRIEF DESCRIPTION OF THE DRAWINGS

FIG. 1 depicts images of acrylonitrile butadiene styrene (ABS) master molds with and without an acetone vapor treatment, as well as polydimethylsiloxane (PDMS) membranes formed using the ABS molds, according to at least some embodiments disclosed herein.

FIG. 2 depicts images showing an effect of a material (e.g., ABS, ABS with an acetone vapor treatment, or polyethylene terephthalate glycol (PETG)) used for 3D printing of a mold on the PDMS membrane formed by casting on the mold, according to at least some embodiments disclosed herein.

FIG. 3 depicts images showing complex patterns created on the PDMS membrane by controlling a pattern at which a material is deposited during a three-dimensional (3D) printing process, according to at least some embodiments disclosed herein.

FIG. 4 depicts images showing the effect of treatment on cell attachment improvement between a PDMS membrane, a PDMS membrane treated with tannic acid or lignin solution, and cells grown in a control group, according to at least some embodiments disclosed herein.

FIG. 5 depicts images of macro-scale patterns created by 3D printing using Fused Deposition Modeling (FDM) on the mold and transferred to PDMS membrane, and their effects on alignment of the cells grown on the PDMS membrane according to at least some embodiments disclosed herein.

FIG. 6 depicts images showing different steps of sheet formation using the silicone-based membranes and preserved cell alignment in stand-alone cell sheets, according to at least some embodiments disclosed herein.

FIG. 7 depicts images of a PDMS membrane used for cell culture and reused for three times after being autoclaved and washed, according to at least some embodiments disclosed herein.

FIG. 8 depicts images of rabbit primary myoblasts, bovine primary myoblasts, and human HepG2 hepatocytes grown on treated PDMS membrane at both low and high confluency, according to at least some embodiments disclosed herein.

FIG. 9 depicts images showing creation of multiple cellular fibers from sheets that are stacked on top of each other and are adhered to one another, according to at least some embodiments disclosed herein.

FIG. 10 depicts images of a first mechanical stimulation scenario and a second electrical stimulation scenario, according to at least some embodiments disclosed herein.

FIG. 11 depicts a schematic diagram of grooves and concave shapes created from patterns on a surface of the silicone-based membrane such that these and concave shapes are fillable with different cell types and cell culture media for purposes of positioning different cell types, according to at least some embodiments disclosed herein.

FIG. 12 depicts A: a chart showing contact angle of a PDMS surface after various treatments; B: a photograph of a droplet of water on PDMS without treatment; and, C: a photograph of PDMS treated with 50 mg/mL tannic acid for 72 hours.

FIG. 13 shows SEM images of a 3D printed mold made with ABS (Left) and the ABS mold treated with acetone vapor for 30 minutes (Right).

FIG. 14 shows higher magnification SEM images of a patterned membrane (left) and the cross-section SEM of the membrane showing the depth and width of the grooves created due to 3D printing features.

FIG. 15 shows low (Left) and high magnification (right) SEMs of decellularized sheets from rabbit cells grown on patterned membranes. Directionality of ECM components can be detected.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

Exemplary Process

The present invention provides a method or process to create cell sheets, optionally with high extracellular matrix (ECM) content, optionally while controlling cell alignment. It should be appreciated that the cell sheets described herein can be made with any cell type from any species. The technique of the present invention is simple, easy to perform, has a low-cost, and uses non-toxic and biocompatible, optionally food-grade and food-safe, materials. Further, the present invention allows for cell alignment, optionally in sophisticated or selected patterns, using a simple molding process optionally with 3D printed molds, optionally prepared with cheap open-source 3D printers and using different types of filament materials. Alternatively, similar patterns can be created on the mold using CNC machining or laser engraving on different materials. In some examples, the present invention provides for the reuse of membranes, for example by autoclaving or a solvent (e.g. isopropyl) washing step or a combination thereof. In some examples, the present invention provides multi-layer cell constructs and induced ECM production with optional ECM crosslinking resulting in strong sheets. The method may use biocompatible, optionally food-grade or food-safe, materials. In some examples, sheets are formed in a short process (518 days). In some examples, sheets can be detached partially or completely using scraping.

In some embodiments, a general method includes three steps. First, the method includes growing cells on a treated elastomeric membrane, such as PDMS or PBAT. PDMS treatment has been done with a variety of materials to make it suitable for cell culture, but these materials are usually expensive or toxic or require multistep treatments, including activation of the PDMS surface. To remedy this concern, the present invention may utilize direct treatment of PDMS or another elastomeric membrane with a polyphenol solution. In some examples, the polyphenol solution is a tannic acid solution in water, or a lignin solution in water and/or methanol or another alcohol, in order to make the membrane suitable for cell attachment. It should be appreciated that both tannic acid and lignin are food safe, natural, and non-toxic materials. These treated membranes can be used for growing cells. The treated membranes can also be washed and autoclaved in order to reuse them for growing new cells. The treatment is stable and no re-treatment is required after autoclaving.

Second, the method includes aligning the cells on a surface of the treated membrane. Cell alignment on different membranes have been performed by creating micron or nanoscale features on the surface using lithography, or by creating protein patterns on the surface of the membrane. However, these processes are expensive, lengthy, and require specialized equipment. Some of the materials used in these processes can be animal-derived and therefore lower the consistency of the process. Distinctly, the present invention utilizes a textured mold to provide surface features on a molded membrane. In some examples, the mold is made by 3D printing, optionally with cheap and open-source printers, or alternatively CNC machining or laser engraving, in order to create micron, nano, or meso-scale patterns that can be replicated to the membrane by casting. This one step pattern creation on the membrane surface is enough to control cell alignment.

Third, the method includes cell sheet formation. Cells are grown and align on the membranes. The membrane patterns also induce ECM formation. Optionally, the cells may be induced to produce higher amounts of ECM by using different growth factors and elements, such as ascorbic acid or by macromolecular crowding. Once the cells are completely confluent and have produced enough ECM, more cells are grown on this cell-ECM layer. Optionally, in order to make a more stable structure, the ECM can be crosslinked with low concentrations of tannic acid or lignin or other crosslinkers that can crosslink different ECM components such as transglutaminase. This can be repeated at least 3 times in a 2 to 3 week time span. At the end of the process, the layers (including the cells and their ECM) can be physically removed (e.g. scraped) from the membrane to make a coherent cell sheet. Other techniques for making cell sheets require either changing membrane properties to make it suitable for cell sheet detachment, digesting some of the proteins that cells need for remaining attached to the surface, or inducing cells to show contraction that forces them to lift off. Distinctly, the method described herein provides a cohesive multilayer cellular construct that can be, for example, scrapped off using an external object. Among other benefits, in at least some examples the process described herein can be used with any adherent cell type from any species.

Optionally, one or more aspects of the method may be used alone or in other combinations. For example, cell sheets may be grown on smooth (i.e. non-patterned) membranes without cell alignment. In other examples, alternative membrane materials may be used that do not require surface treatment. In other examples, membranes may be patterned by machining or other physical processes applied to a solid membrane.

Membrane Fabrication

As used herein, the term “membrane” refers to a substrate suitable for supporting a cell sheet. Porosity is optional, but the membrane is typically a bulk material. The membrane is non-porous to bulk liquid flow or at least capable of supporting a liquid media over the cells. Membranes may be made of various thermoplastics or thermosets. The membranes may be rigid or elastomeric. Rigid membranes can be molded, for example, from biocompatible epoxies. Elastomeric membranes can include, for example, thermoplastic elastomers based on poly(butylene adipate-co-terephthalate) (PBAT), and urethane rubbers formed by reacting a polyol with an isocyanate. Optionally, commercially available materials from Smooth-On, Polytek, and Reynolds Advanced Materials can be used for cell culture related applications. Elastomeric membranes may also be made of a silicone, for example PDMS. However, elastomeric membranes typically have limitations due to their hydrophobicity, such as inappropriate cell attachment, agglomeration, and detachment, which may be improved with surface treatment as described herein. PDMS is a preferred material and is used as an exemplary membrane material in many examples herein, but other materials may be used.

A method of fabricating an elastomeric membrane, such as a PDMS membrane, is also described herein. It should be appreciated that PDMS comprises an elastomer base and an elastomer curing agent. Other silicone-based or non-silicone-based elastomers have two components that upon mixing will catalyze their reaction into forming elastomeric resins or rubbers. The method includes several process steps, such as: mixing the elastomer base and the elastomer curing agent or the two components of the elastomer at certain ratios (e.g., a 1:1 to 1:20 volume or weight ratios of the curing agent to the base) and casting and heating the elastomeric solution to cure the polymer and form the membrane. Methods of 3D printing may be used to print molds that are then used for casting the elastomeric solution and creating the membranes. An example 3D printing method that may be used includes Fused Deposition Modeling (FDM) 3D printing. Patterns on the surface of the 3D printed molds (which may be or include patterns inherent to the 3D printing process) may then be mirrored on the membrane. This may be leveraged to create patterns (e.g., parallel patterns) on the elastomeric membrane that can be used for aligning the cells.

Different polymeric materials can be used with different commercially available 3D printers, such as UltiMaker and Prusa, for printing the molds. Alternatively or additionally, other techniques of creating patterns such as CNC machining or laser engraving, may be used to make a mold or modify a 3D printed mold. A 3D printer may be configured or operated to create patterns with different resolutions that can have different effects on cell alignment. When 3D printers are used for printing the molds, nozzle sizes can be 0.1-1 mm, for example 0.25 mm, 0.4 mm, or 0.6 mm, and the printing speed can be 20-100 mm/s. It should be noted that smaller nozzle sizes and lower printing speeds will create smaller features that might perform better in cell alignment depending on the cell type and its sensitivity to the environmental conditions. In some examples, thermoplastic polymers, such as PETG, ABS, and PVB (polyvinyl butyral) are used in 3D printing. However, it should be appreciated that other polymers may be used that are not explicitly listed herein. Further, other methods can also be used to create molds using non-polymeric materials, such as metallic molds. The molds may be optionally treated to alter the patterns. For example, ABS molds may be treated with acetone vapor and PVB molds may be treated with isopropyl alcohol vapor to melt the surfaces of the polymeric mold and change the resolution of the 3D printed part and consequently change the size or smoothness of the patterns on the membrane. It should be appreciated that timing of the treatment defines a final pattern or lack thereof.

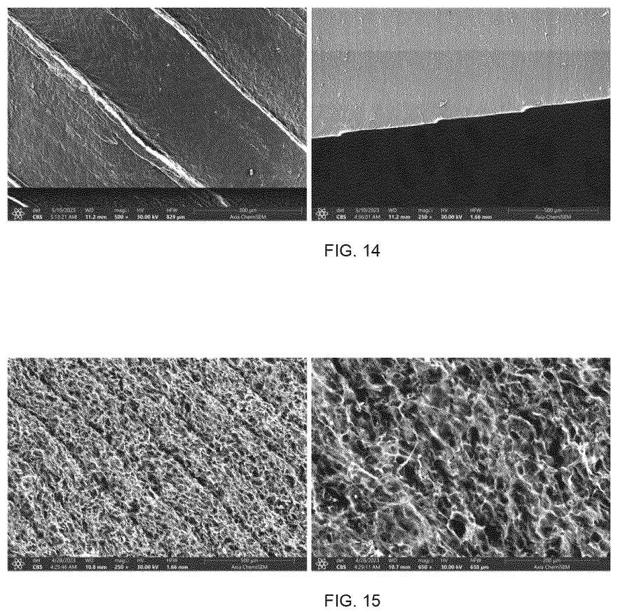

Independent of the material used for 3D printing the molds, 3 levels of features may be created on the surface of the mold. The meso scale patterns, larger lines seen in FIGS. 13 and 14, can have 100-500 μm spacing. These patterns can have some moderate effect on cell alignment. The micron scale patterns, thinner lines spaced 5-50 μm aprat, will be most important in aligning the cells. Nano scale patterns, features smaller than 1 μm create surface roughnesses that induce cells to secrete higher amounts of ECM components. These features together create a 3D-like environment for cells growth and function as compared to surfaces with no features that are completely two dimensional and by creating a basolateral morphology, change the cells' behavior compared to in vivo conditions. Using larger sizes of printer nozzles or higher speeds of printing will shift the feature sizes to larger ones in the mentioned range.

Other methods such as using sandpapers with different grits can also be used to change the features on the surface of the mold after printing but they can create new extra features while eliminating the old ones. Such methods might not be as reproducible as treatment with solvent vapor.

In some examples, treatment times with the vapor phase of a solvent of a polymeric filament 3D printed mold ranged from about 0 minutes to about 30 minutes. After the vapor treatment, the molds can be heat treated at temperatures 20-60° C. for 1-12 hours in order for the solvent to evaporate from the surface of the mold and for the polymer to solidify. When the molds are ready, the elastomer solution may be cast and heated at a temperatures ranging from 25-90° C. for a time period of 1-24 hours to allow the polymer to cure. Performing the heating process at higher temperatures will result in less time required for solidifying the elastomeric resin.

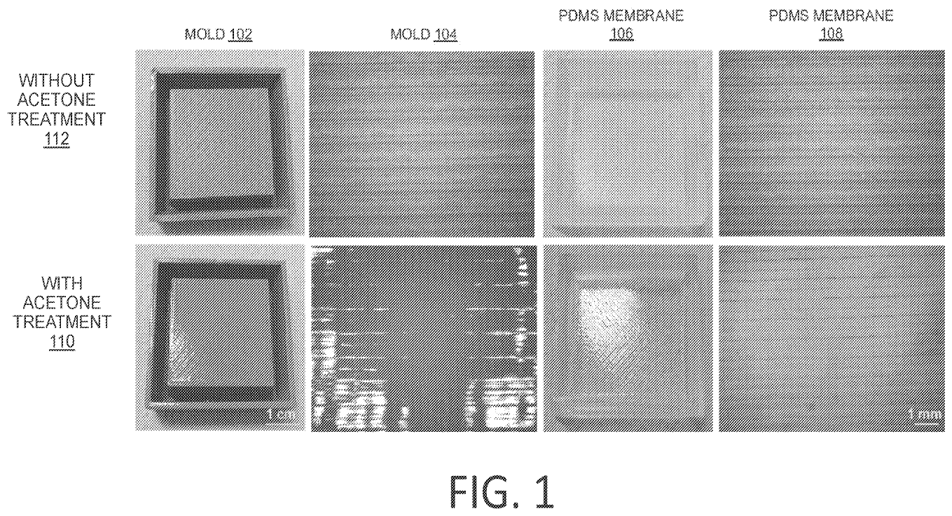

FIG. 1 depicts images of ABS molds with and without an acetone vapor treatment, as well as PDMS membranes formed using the ABS molds, according to at least some embodiments disclosed herein. Specifically, FIG. 1 depicts a first row 112 associated with the ABS molds 102, 104 and the PDMS membranes 106, 108 formed using the ABS molds 102, 104 without the thirty-minute acetone vapor treatment and a second row 110 associated with the ABS molds 102, 104 and the PDMS membranes 106, 108 formed using the ABS molds 102, 104 with the thirty-minute acetone vapor treatment.

In the examples shown, molds 102 have a platform 110 and a moat 112. Membrane 106 has a bottom 116 and a wall 118. In the examples shown, the wall 118 is polygonal with multiple segments. In other examples, the wall 118 may be of another shape, for example round. In FIG. 1, the membrane 106 has been removed from the mold 102 and inverted. An upper surface of the bottom 116 is formed by resin cast on the platform 110. The wall 118 is formed by resin cast in the moat 112.

The photograph of mold 104 shows an enlargement of a region of the platform 110 of the photograph of mold 102. The platform 110 has a topographical pattern, in this example produced inherently by a process of 3D printing and optionally, as in the case of second row 110, with further modification. In at least the region of platform 110 shown in the photograph of mold 104, the pattern includes a set of generally parallel grooves 110. The photograph of membrane 108 is an enlargement of a region of the bottom 116 of the photograph of membrane 106. The upper surface of the bottom 116 of the membrane 106, 108 has a topographical pattern mirroring the topographical pattern of the platform 110 of the mold 102, 104. In at least in the region shown in the photograph of the membrane 108, the upper surface of bottom 116 of membrane 106, 108 has a set of generally parallel grooves 120. The wall 118 may be, for example 3-10 mm high, and can contain a liquid media and cells prior to attachment on the bottom 116 of membrane 106, 108. A first layer of the cells attaches, preferably temporarily, to the bottom 116 of membrane 106, 108. In the example shown, the membrane 106, 108 is square in plan view and about 30 mm wide and long. Optionally, the membrane 106, 108 may have other shapes, for example round or rectangular. The membrane 106, 108 may also have other sizes, for example a length or diameter of 10-100 mm or more.

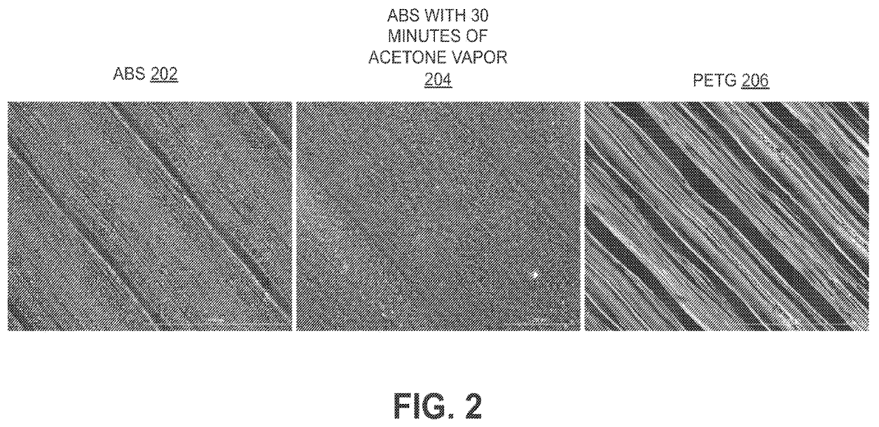

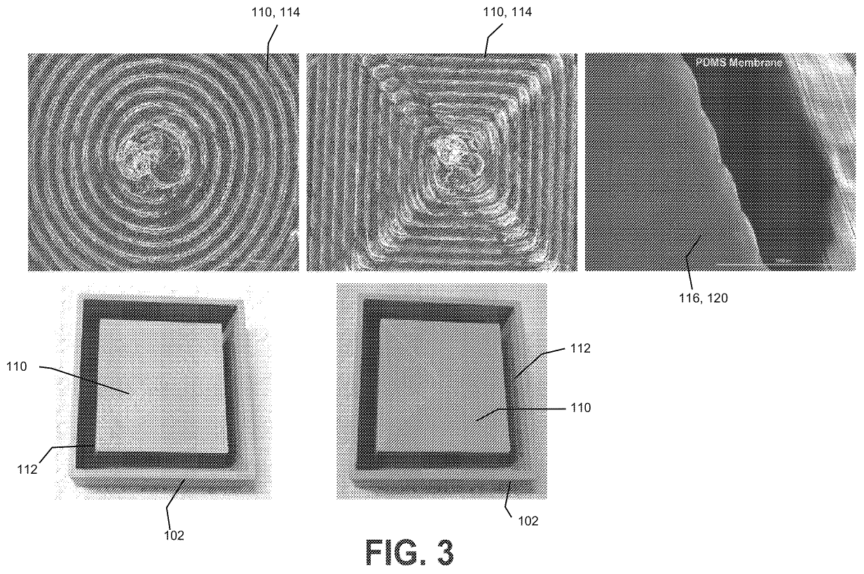

FIG. 2 depicts images showing an effect of a material (e.g., ABS 202, ABS with an acetone vapor treatment 204, or PETG 206) used for 3D printing of a mold, according to at least some embodiments disclosed herein. The present invention also contemplates creating patterns, such as circular or concentric patterns, by controlling the pattern at which the material is deposited during the 3D printing process, such as the ones shown in FIG. 3, among others not explicitly listed or depicted herein.

It should be appreciated that independent of the pattern or material used for printing, the cross-section of the pattern, e.g. grooves of the pattern, is typically similar. Other methods and materials can also be used for making the molds, preferably wherein the size of the patterns (e.g. the spacing between grooves) is similar to what is created using 3D printing, or other sizes that can have similar effects on attachment and alignment of the cells or ECM production. For example, CNC machining and laser ablation of different materials may be used to make similar molds. Furthermore, the molds can be made from metallic or polymeric materials, as well.

Treatment of Elastomeric Membrane Surface for Cell Attachment

Many elastomeric materials, including silicones and many non-silicone-based membranes, are hydrophobic materials. Hydrophobic materials are generally unsuitable for cell attachment and tend to cause cells to form aggregate clumps rather than sheets. Membrane fabrication to impart surface topography as described above may improve cell sheet formation. Further, the surface of elastomeric or other membranes are optionally modified by chemical treatment, e.g. with a hydrophobic agent, to make them more suitable for cell attachment.

In some examples, elastomeric membranes, for example silicone membranes such as PDMS or PBAT, are treated with an aqueous solution of a polyphenol. The polyphenol may be plant based, for example tannic acid or lignin. The polyphenol may be applied to the membrane without additional cross-linkers or compounding agents, for example iron.

In some examples, the treatment may include contacting the membrane with 10-200 mg/mL of tannic acid, optionally 25-100 mg/mL. The treatment may be done generally at ambient or room temperature. The contact period may be about 6 hours to 4 days, or 1-3 days. The tannic acid solution may be essentially unbuffered, for example, the aqueous solution may have a pH of 7.5 or less or 7.0 or less. Optionally, the tannic acid solution contains only tannic acid and water. The tannic acid is applied to the membrane without additional cross-linkers or compounding agents, for example no iron is present. Without intending to be limited by theory, dihydroxyphenol or truhydroxyphenol groups of the lignin may be able to adsorb to the surface of PDMS or other elastomers even without surface activation of the membranes. After optional treatment with a caustic solution, as described further below, the membrane may also have functional groups (e.g. silanol groups) available for chemical binding. However, surface activation is not required. Further, additional cross-linking of the polyphenol (e.g. with iron or polyethyleneimine, or beyond any crosslinking that may be present in the natural molecule) is not required. In some examples, a cell growth substrate is produced that has essentially only the membrane material and attached polyphenol molecules, wherein the polyphenol molecules are not bound to each other except via the membrane.

In some examples, the treatment may include contacting the membrane with a solution of about 0.1-6 mg/mL, or 1-4 mg/mL of lignin. The treatment may be done generally at ambient or room temperature. The contact period may be about 6 hours to 4 days, or 1-3 days. The tannic acid solution may be essentially unbuffered. Optionally, the tannic acid solution contains only tannic acid, water and a water-miscible solvent, for example methanol or another alcohol.

It should be appreciated that lower or higher concentrations of polyphenol can be used for this purpose as well, but the timing of the treatment should be adjusted accordingly. In some embodiments, a solution containing both tannic acid and lignin can be used, or different solutions containing tannic acid and lignin can be used one after the other.

In order to improve the attachment of tannic acid, lignin or another polyphenol to the surface of the membrane, the membrane can be pretreated with a caustic solution, for example an aqueous sodium hydroxide solution. Alternatively, the membrane may be pretreated with an oxygen, atmospheric air or carbon dioxide plasma. Optionally, the membrane is not pre-treated with either the caustic solution of plasma.

After the polyphenol treatment (i.e. with tannic acid and/or lignin), the membranes may be washed to remove residues. For example, the membranes may be washed with deionized water, optionally at least twice, to remove the residues. Then, the membranes may be used immediately for cell culture or can be stored at room temperature for a time period of a few weeks or in a refrigerator for a longer time period.

For sterilization purposes, before starting the polyphenol treatment, the membranes can be washed with about 70% ethanol and subjected to UV afterwards or they can be autoclaved. The polyphenol (i.e. tannic acid and/or lignin) solutions can be sterile-filtered using about 0.2 μm syringe filters. The treatment may be done inside a biosafety cabinet to avoid possibility of contamination.

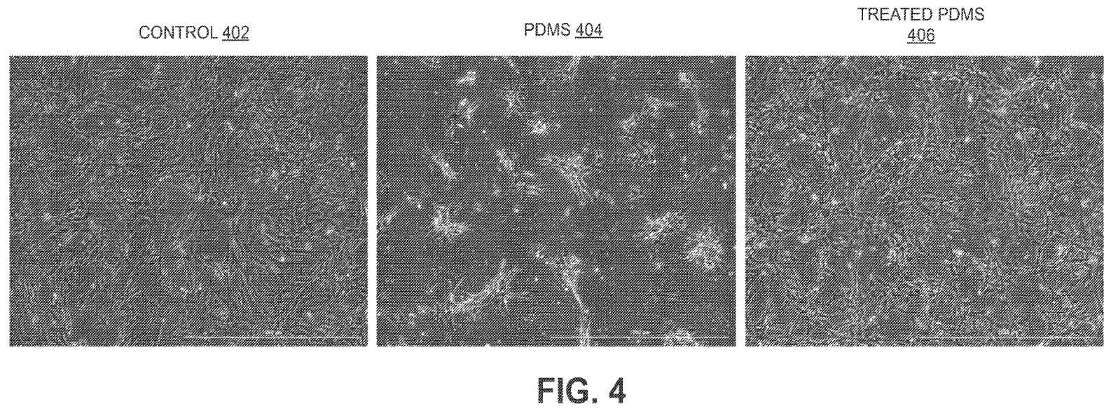

FIG. 4 depicts images showing the effect of treatment on cell attachment improvement between a PDMS membrane 404, a treated PDMS membrane 406, and cells grown in a control group 402 (e.g., a commercially available cell culture treated polystyrene), according to at least some embodiments disclosed herein. As shown in FIG. 4, the PDMS membrane 404 without treatment does not allow for proper cell attachment and cells will either form agglomerates and clumps or will die. The membranes here don't have any patterns and as such cells are not aligned. Optionally, smooth membranes can also be used for making cell sheets but without any intentional cell alignment.

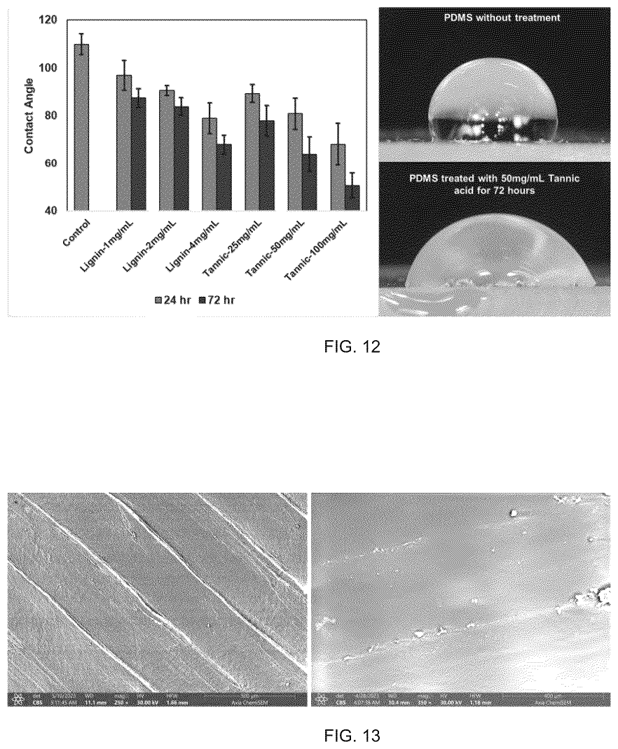

FIG. 12 shows the effect of concentration and time in various examples of treatment with tannic acid or lignin on the hydrophilicity of PDMS membranes as measured by their contact angle. The PDMS membranes were not pre-treated to activate their surfaces, for example with a caustic solution or plasma. Three concentrations of each of the tannic acid (25, 50, and 100 mg/mL) and lignin (1, 2, and 4 mg/mL) aqueous solutions were used with either 24 or 72 hours of treatment. In each case, three 1×1 cm pieces of PDMS membrane were cut and treated at room temperature. Contact angle measurement was performed by taking pictures of 10 μL droplets of water on each membrane using a portable digital microscope with 50-1000× magnification. Pictures were analyzed using Contact Angle Plugin in ImageJ. For both tannic acid and lignin, increasing the concentration or duration of treatment decreased contact angle. In cell growth experiments, membranes with contact angles in a range of about 60-87 degrees have been used to grow cell sheets. However, better results are obtained with lower contact angles near 60 than with contact angles near 87. Further, literature suggests that contact angles of 55, or even down to 20, may be useful. Accordingly, the concentration and duration of treatment are optionally chosen to produce a contact angle of 87 degrees or less, 85 degrees or less, 80 degrees or less, 75 degrees or less or 70 degrees or less. The concentration and duration of treatment are optionally chosen to produce a contact angle of 20 degrees or more, 40 degrees or more, 55 degrees or more or 60 degrees or more. The preferred contact angle may also vary with cell type or surface topography of the membrane. Treatment with 50 mg/mL solution of tannic acid for 72 hours has produced high quality cell sheets using a variety of cell types and with smooth or textured PDMS membranes.

The treated membrane is hydrophilic but not coated or functionalized with exogenous ECM components (i.e. ECM components not made by cells grown on the membrane) such as structural proteins (e.g. collagen, elastin, fibronectin and laminin), proteoglycans and glycosaminoglycans. While adding ECM components to the membrane encourages cell attachment, this form of attachment may be too strong for cell sheet detachment. Further, in at least some examples, it is desirable to avoid all use of exogenous ECM components and to instead produce cell layer or a cell sheet that contains only ECM made by the cells grown on the membrane. Similarly, in some examples the contact angle of the treated membrane is moderate, for example 40 degrees or more 55 degrees or more, since a lower contact angle may result in a cell construct that is more difficult to separate from the membrane.

Cell Alignment

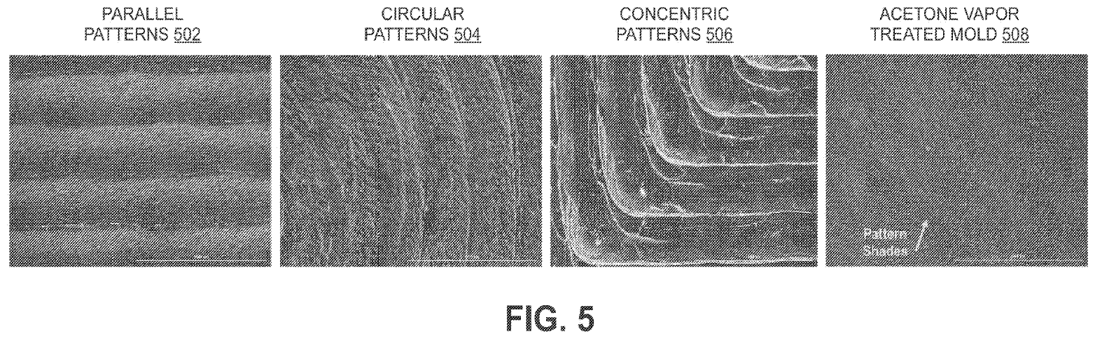

It should be appreciated that 3D printing using FDM has a very low resolution and creates meso-scale patterns (up to a few hundred microns) as well as other micron and nano-scale patterns. Although these patterns can be considered as artifacts, they can be used as topographical signaling cues for cells that are much smaller to be aligned in certain directions, as shown in FIG. 5. Specifically, FIG. 5 depicts parallel patterns 502, circular patterns 504, concentric patterns 506 and pattern shades of an acetone vapor treated mold 508. By controlling the pattern of printing in different locations of the mold, a membrane with different patterns in different locations can be created, which can then be used to pattern cells. An example of this is in the case of skeletal muscle tissue where all of the cells should be aligned to properly differentiate and form muscle fibers that are aligned with each other. As an illustrative example, rabbit primary myoblast cells are used for demonstration purposes in FIG. 5. In case of molds treated with ABS, treatment with acetone vapor for some periods of time (e.g., 5-30 minutes or more) can result in diminishing the patterns on the mold completely or to a degree that cells won't be able to understand them. As a result, cells still show proper attachment but without any alignment.

Cell Sheet Formation

As described herein, a “cell sheet” is a scaffold-free or self-assembled sheet-like construct including cells and the ECM that they have secreted, or a portion of such a construct. Cell attachment to each other and to their ECM is preserved in the cell sheet and therefore the cells sheet can be maintained independent of a membrane or surface to adhere to at least for a period of time. Since the ECM is preserved in each sheet, if multiple sheets are stacked on top of each other, they can bind and fuse to one another or to tissues or organs.

Further, elastomeric or other membranes, with or without patterns, and optionally treated with lignin and/or tannic acid, can be used for sheet formation in a simple process. The membrane provides a proper surface for cell adherence and cells form strong attachments to the membrane at the beginning. Over time, cells proliferate and start adhering to each other and form strong cell-cell junctions. They will also secrete their own ECM, which they will then adhere to.

As cells become more confluent and establish strong attachments to each other or their ECM, and as more layers are added as described below, the attachment between the cells and the membrane weakens. Once the attachment of cells to each other and their ECM becomes stronger than their attachment to the membrane, cells and their ECM can be detached, or detachment can be initiated, by a scraping method. It is also possible to make a cell sheet by scratching the edges of the membrane and shaking it to help cells delaminate as a sheet by applying shear force. It should be appreciated that the sheets are also strong enough to be grabbed and pulled off of the surface using a tweezer or similar means. Once pulled off from the surface, cells will maintain their alignment due to the high density of the cells and high ECM content in the sheets that prevents them from changing their alignment for a period of time. Optionally, the membrane can be scraped to release edges of cell sheets, followed by a) pulling the cell sheet edges to form or release the rest of the cell sheet, b) waiting for a period of time, for example 0.2 to 2.0 hours and then scrapping or pulling to form or release the rest of the cell sheet, and/or c) waiting optionally while continuing to incubate the cells wherein the rest of the cell sheet may be formed or released spontaneously. Separating the lower layer of the cells from the membrane may cause a nearly instantaneous change in the bottom surface of the separated cells of the lower layer towards a detached sheet structure, and may trigger further changes in any cells that are still attached to the membrane. With an elastomeric membrane, scraping the membrane may also deform parts of the membrane that are not directly scraped, thus causing or helping with detachment of cells attached to parts of the membrane that are not directly scraped. A cell sheet may include a complete construct fully released from a membrane, or an edge or other portion of a construct that has been released from a membrane while another part of the construct remains attached to the membrane.

A cell sheet may have one or more layers. Once the cells cover an entire surface of the membrane and produce ECM, it is possible to add more cells in order to grow on the initial layer of cells and create a multilayer construct. This can be performed multiple times to make a thicker construct, but if it is done more than a certain number of times, depending on the cell types and species up to 10 times, the attachment between cells of different layers becomes much stronger than attachment of the first layer to the membrane, which could result in spontaneous detachment of the cells from the membrane. While spontaneous detachment may be useful, particularly if instigated by scraping, scratching or shaking, uncontrolled spontaneous detachment of the cell layers may be followed by excessive shrinkage that may rapidly turn the sheet into an agglomerate.

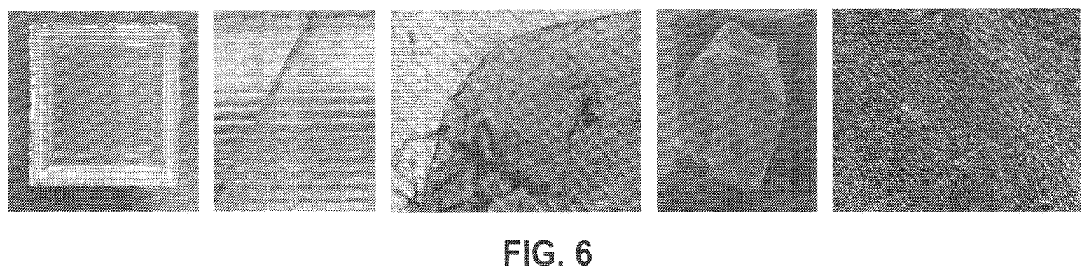

Further, it is possible to induce cells to secrete more ECM components by treating them with components, such as ascorbate family members, or by creating macromolecular crowding by addition of elements such as high molecular weight PEG or carrageenan with proper concentrations to the media. It is also possible to crosslink the ECM secreted by cells prior to addition of more cells to increase stability of the cellular constructs. Lower concentrations of tannic acid or lignin (e.g. less than 0.1 mg/mL tannic acid or less than 10 μg/mL lignin) for short amounts of time in serum-free media to avoid agglomeration of media components can be used for crosslinking ECM components. The timing of treatment can be in a range from about 5 minutes to about 100 minutes depending on the crosslinker type and its concentration prior to addition of the new cells. Multiple cell layers, as well as more ECM content and crosslinking, can result in a sheet with better mechanical integrity that can help with handling the sheets and moving them from one vessel to another after delamination. Additionally, the present invention contemplates use of multiple cell types either in each layer or different cell types on different layers. For example, FIG. 6 depicts images showing different steps of sheet formation using the PDMS membranes and preserved cell alignment in stand-alone cell sheets, according to at least some embodiments disclosed herein.

In an exemplary method, on Day 1, growth media is added to a depth of about 3-5 mm over the membrane. Enough cells are added to over the membrane to about 60-90% or 75-85% confluence. The cells are incubated, for example at 37 C or the physiological temperature of the cell animal, for 2 days. On Day 3, the cells will have grown to form a completely confluent first (single cell) layer. The growth media is refreshed, i.e. removed (e.g. by aspiration) and replaced with fresh media. In the case of stem cells, the fresh media may be a differentiation media. For stem cells, differentiation and fusing to adjacent cells typically happen together. For other cell types that adhere (rather than fuse) to each other, the fresh media may be more growth media.

On Day 4, enough cells are added to cover the first layer of cells to about 60-90% or 75-85% confluence. On Day 5, the media is refreshed, with either new growth media or new differentiation media for stem cells. On Day 6, a second confluent layer has formed and enough new cells are added to cover the second layer of cells to about 60-90% or 75-85% confluence. On Day 7 the media is refreshed, with either new growth media or new differentiation media for stem cells. On Day 8, a third confluent layer has formed and enough new cells are added to cover the third layer of cells to about 60-90% or 75-85% confluence. On Day 9 the media is refreshed, with either new growth media or new differentiation media for stem cells. On Day 10, a fourth confluent layer has formed. In this example, the cell sheet will have four layers. Optionally, more or less, i.e. 2 or 3 or 5 or more, layers may be produced. On Day 11, and every two days thereafter, the media is refreshed again. The cells continue to produce ECM and the cell sheet becomes more robust. On Day 18, an edge of the cell sheet may be scrape from the membrane, and the cell sheet may be pulled away from the membrane. This example describes a typical process but the schedule of cell and media addition or various other steps may be varied.

Reusing Molds

Once a cell sheet is delaminated from the membrane, the cells and their ECM are removed. Although the cell sheet is removed, the membrane could allow unspecific penetration of certain smaller molecules to its bulk, which renders the membrane opaque. It is possible to first remove most of these components by submerging the membrane in deionized water and autoclaving the membrane, for example for a time period of about twenty minutes. To further remove such residues, after autoclaving is performed, the membrane may be submerged in isopropyl or other non-polar solvents that can cause swelling in elastomeric membranes, for example for a time period of about 10-60 minutes. The solvent will swell the membrane and the rest of these residues may be removed. At this point, the membrane can be used for cell culture again without repeating the treatment with tannic acid and/or lignin.

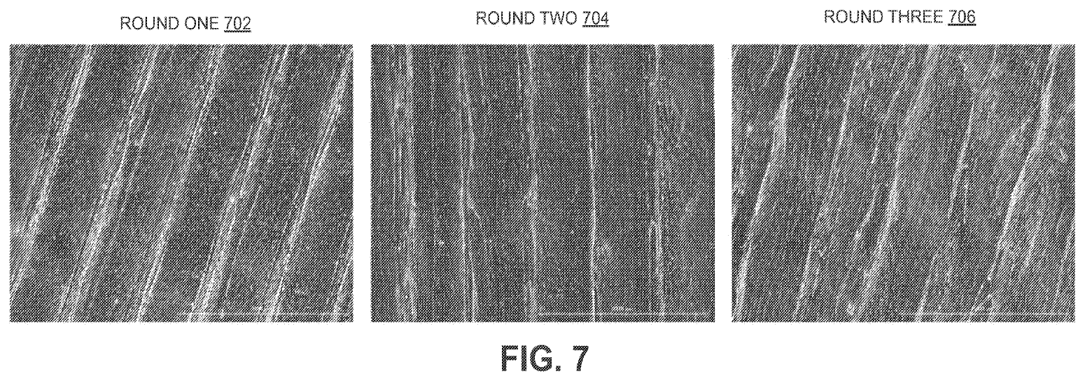

As an illustrative example, FIG. 7 depicts images of a PDMS membrane used for cell culture and reused for three times, round one 702, round two 704, and round three 706, according to at least some embodiments disclosed herein. There are no apparent differences between cell attachment, growth, differentiation or alignment. Further, rabbit myoblasts were grown and differentiated to muscle fibers on the PDMS membrane for a time period of about 8 days in each round (e.g., the round one 702, the round two 704, and the round three 706).

Cell Types

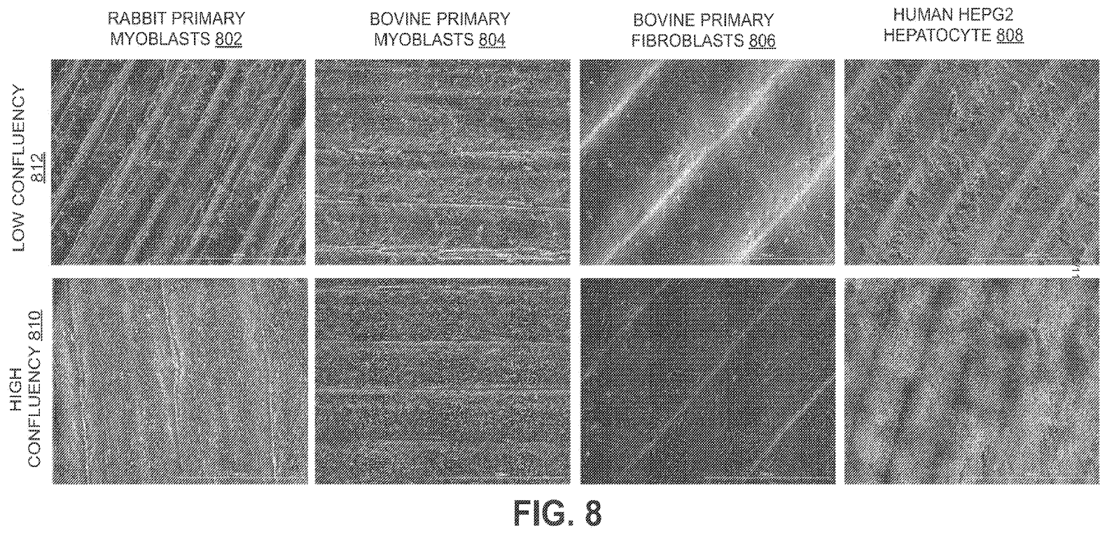

The membranes, for example tannic acid/lignin treated elastomeric membranes with or without patterning, can be used for growing different cell types from different tissues of different species, for both primary and immortalized cells. FIG. 8 depicts images of rabbit primary myoblasts 802, bovine primary myoblasts 804, bovine primary fibroblasts 806, and human HepG2 hepatocytes 808 at both low confluency 812 and high confluency 810, according to at least some embodiments disclosed herein. Different stem cells such as adult stem cells, embryonic stem cells, or induced pluripotent stem cells can be used in the process as well. The stem cells can also be differentiated to the target cells while grown and maintained on these membranes by switching the culture medium from growth medium to proper differentiation medium. As described herein, “confluency” is the percentage area covered by adherent cells. This measurement is routinely used to monitor cell growth and expansion during cell culture experiments. It is useful in determining the optimal timings for cell harvest, passage and process interventions such as drug treatment or cell differentiation.

Specifically, according to FIG. 8, myoblasts that have elongated spindle-shaped morphologies from both rabbit and bovine sources (e.g., the rabbit primary myoblasts 802 and the bovine primary myoblasts 804) showed alignment in accordance with the membrane patterns, while bovine fibroblasts (e.g., the bovine primary fibroblasts 806) maintained their flattened morphology. Hepatocytes with round morphology (e.g., the human HepG2 hepatocytes 808) also maintained their shape independent of the membrane patterns.

Assembly of Sheets

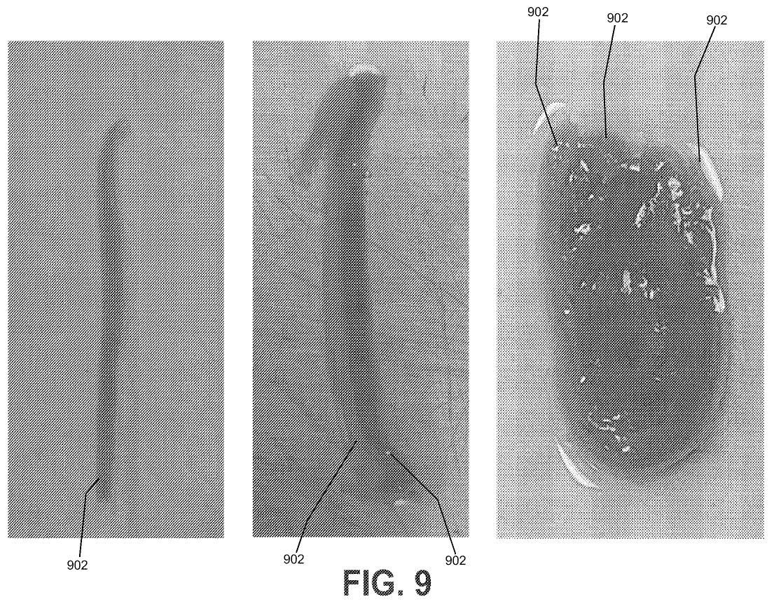

In some examples, the cell sheets can be formed or remodeled into cell constructs of other configurations. For example, cell sheets can be rolled on themselves, and preserved ECM will result in formation of a coherent cellular structure that cannot be unrolled. Multiple cellular fibers 902 made out of rolled sheets can be stacked on top of each other, and they will also form firm attachment to each other, as shown in FIG. 9. If skeletal muscle cells are used, the rolling can be done in a direction that results in the muscle cells aligned in the direction of the larger fiber. This functioning unit can be used for different applications, such as in vitro modeling of skeletal muscle tissue for drug discovery or modeling diseases, development of cultivated meat, or as a building block for biofabrication of skeletal muscle tissue for regenerative medicine applications.

The present invention also contemplates creating cellular fibers with different cell types and stacking them. Examples of this application include creating marbling for cultivated meat by using skeletal muscle, adipose, and connective tissue derived cells, or creating more physiologically relevant models of different tissues and organs by recreating cellular crosstalk. Sheets stacked on top of each other without first rolling them, will also adhere to each other and form a coherent structure. An example of this includes making sheets of hepatocytes and stacking them to make liver-like tissue for either in vitro modeling or making foie gras as food.

Bioreactors for Dynamic Environment

Cells constantly receive dynamic cues in in vivo conditions. These cues include electrical and/or mechanical stimulation. Recreating such cues in the in vitro conditions can be used to guide cellular behavior including cell alignment, ECM production, and even differentiation path of the stem cells to different cell types. It is possible to apply mechanical stimulation to the cells grown on an elastomeric membrane by stretching or compressing the membrane that is flexible. Electrodes can also be inserted inside the bulk of the membrane close to the surface or above the surface to apply electrical stimulation to the cells. FIG. 10 depicts a first mechanical stimulation 1002 scenario and a second electrical stimulation scenario 1004. For mechanical stimulation, a membrane 106 is attached to a linear actuator 1006. The linear actuator 1006 is adapted to compress or stretch the membrane 106. For electrical stimulation, electrodes 1008 are cast in a membrane 106. The electrodes 1008 are attached to a power supply 1010 adapted to provide a an electrical voltage and/or current to the electrodes 1008.

Different parameters can be controlled to deliver appropriate mechanical or electrical stimulation to the cells depending on the cell type, species, or the specific application. For mechanical stimulation, cells can be exposed to static or dynamic stimulation. In static stimulation, the elastomeric membrane can be stretched for 1-20%. This stretched condition can be maintained for 10-100 minutes or longer time before the elastomeric membrane is returned to its original resting size. After a certain amount of time, a similar or different stretching condition for similar or different amount of time can be implemented. In dynamic stimulation, important parameters are amplitude and frequency of the stimulation as well as the active time. The amplitude of the deformation/stretching can be 1-20% or more and the frequency can be 0.1-10 Hz or more. The stimulation can be performed for 1-10 minutes or more followed by 1-10 minutes or more in rest mode before the same or different stimulation is applied. Similar static or dynamic electrical stimuli can be applied by controlling the voltage (0.1-5V), frequency (1 mHz to 20 Hz or more), as well as duty cycle.

Patterning Different Cell Types

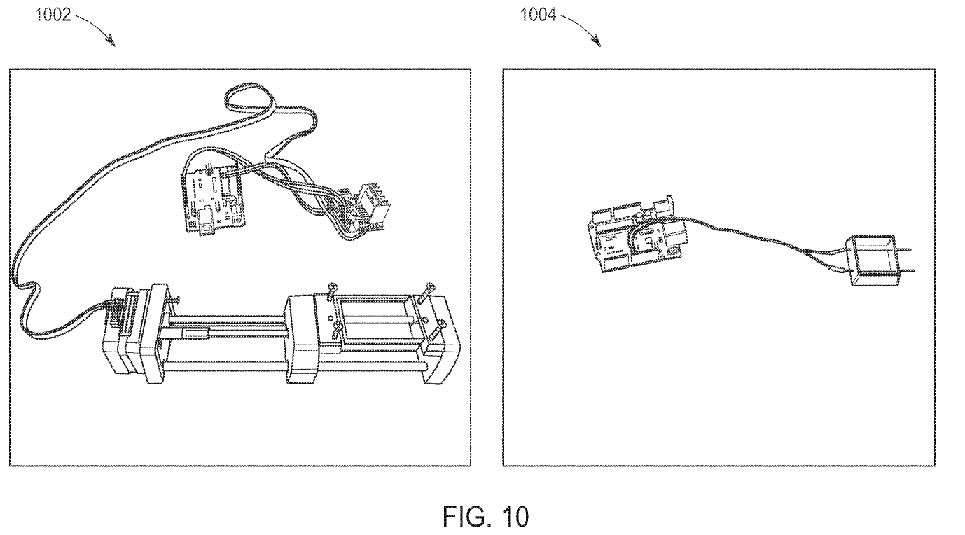

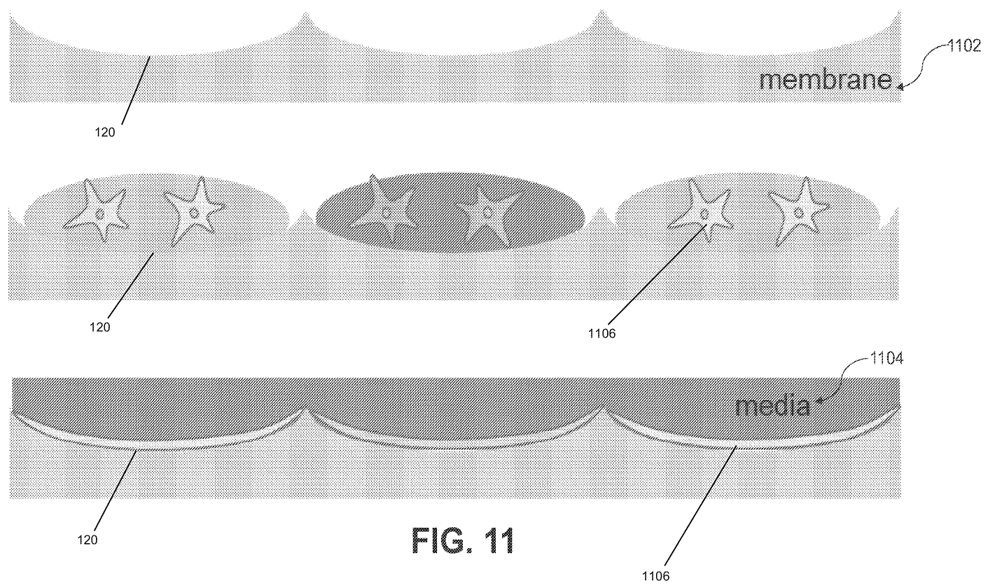

The patterns created on the surface of the membrane 1102 of FIG. 11 creates grooves 120 with concave shapes. Although the depth of these grooves may be not much bigger than the size of the cells 1106 themselves (cell 1106 size is highly exaggerated in the middle panel of FIG. 11) and they can still cover the whole surface uniformly, this feature can be used to pattern or align different cell types on the membrane 1102. Optionally, each of these concave grooves 120 can be filled with a low volume of cell culture media 1104 of FIG. 11 with a certain cell type in a way that the solutions in adjacent grooves 120 do not touch each other. After a short amount of time (e.g., a time period of about thirty minutes to about sixty minutes), cells adhere to the surface of the groove 120 and extra media 1104 can be added to cover the whole surface. This can be used to position different cell types on different locations of each membrane 1102. For example, by patterning fat and muscle cells, it is possible to create marbling for cultivated meat application.

Decellularized Cell Sheets as Fibrous Scaffolding

After the sheets are formed and removed from the membrane, since there is a high content of ECM present, the sheets can be decellularized using mild detergents such as Triton X-100, SDS, or Tween 20, at room temperature for a short time or on ice for longer times. Decellularization can be performed with 0.1-1% V/V or wt/V solutions of each of the mentioned detergents or a combination of them at room temperature or at 4° C. The decellularization can be performed for 1-48 hours depending on the temperature or concentration of the detergent. The solvent for the detergents can be either deionized water or a buffer such as phosphate buffered saline (PBS). It is also possible to treat the sheets with one detergent solution, wash them with deionized water or PBS, and continue the treatment with a fresh batch of similar or different detergent. Using fresh detergent solution can further improve the decellularization process.

After decellularization, a fibrous sheet of rich ECM will remain that can be used for 3D cell culture in vitro or in vivo regenerative medicine and tissue engineering applications. The ECM sheets can also be treated with DNase and RNases to remove any DNA and RNA residues. By using different cell types or treating them with different types of media, the composition of ECM can be controlled. Membrane surface patterns, as well as external stimuli including electrical and mechanical stimuli, can be used to align the ECM components in certain directions, as described herein. Further crosslinking of the ECM proteins, before or after decellularization can also be performed. The directionality of ECM will be preserved during decellularization and can guide cellular behavior after re-cellularization.

Solubilized ECM

The decellularized ECM can also be solubilized to create liquid ECM that can be used for surface treatment or 3D cell culture. The decellularized ECM can be solubilized using a high ionic strength solution, such as 3-4 molar urea solution in deionized water or PBS. For this purpose, decellularized sheets can be submerged in such solutions and kept on ice for 24-48 hours. Every 6-12 hours, sonication with 1-10 Hz frequency for 2-5 seconds can be applied to disrupt the ECM and further ease the dissolution of the ECM molecules in the buffer. The sonication should be implemented while the samples are kept on ice to avoid denaturation of ECM proteins due to the generated heat. Similarly, treating the decellularized sheets with enzymes such as Pronase, Dispase, or different collagenases (such as collagenase type I, II, III, and IV) or their combinations. Depending on the origin of the cells as well as the conditions of ECM production, different concentrations of these enzymes can be used including 0.1 to 2 mg/mL, 0.5 and 5 U/mL, or 0.1 to 5 mg/mL. The timing of the treatment varies between 12 hours and up to 4 days. Sonication and homogenization can be performed to help with solubilization. It should be appreciated that these processes are performed on ice. After solubilization, the solution is filtered using dialysis filters to remove the enzymes or ionic solution. It can then be lyophilized and solubilized again in the deionized water to achieve the desired concentration. This solution can then be kept refrigerated or frozen until use. The potential applications for this include treatment of surfaces that are not suitable for cell attachment to render them cell adhesive or 3D culture systems using “cell-derived ECM” as the main matrix. This solution is temperature sensitive and can be gelled quickly in a few minutes by increasing the temperature to a temperature of about 37° C.

### Example 1: Formation of Cell Sheets with Rabbit Myoblasts on Patterned Membranes Made with Aligned ABS Molds

In this experiment, rabbit myoblasts are differentiated to skeletal muscle cells and used to form a cell sheet with aligned cells, following these steps:
1. A master mold is 3D printed using Original Prusa i3 MK3S+ and ABS. Nozzle size is 0.4 mm and printing speed is 60 mm/s.
2. PDMS SYLGARD 184 is used for making the membranes with a 10:1 ratio of the base and curing agent. Curing is performed at 60° C. for 4 hours. The membrane is 3×3 cm in dimensions.
3. After sterilizing the membrane with 70% ethanol, it is treated with 3 mL of sterile aqueous solution of tannic acid with a concentration of 50 mg/mL for 72 hours, after which it is washed three times with deionized water to eliminate any traces of tannic acid.
4. Rabbit myoblasts (Sigma-Aldrich, RB150-05) were grown up to 80% confluence in 10 cm petri dishes in their growth medium (Sigma-Aldrich, RB151-500). They were dissociated using trypsin and 5×10⁵ of them were added to the membrane in the same growth medium. This is considered day 1 of the sheet formation process and the number of cells will result in an 80% confluency once cells adhere to the membrane.
5. On day 3 of the experiment, medium is switched to the differentiation medium of the cells (Sigma-Aldrich, 151 D-250) plus enough L-ascorbate-2-phosphate to achieve a concentration of 100 μg/mL. This media is refreshed every two days till the end of the experiment.
6. On days 4, 6, and 8, the same number of cells (5×10⁵) were added on top of the membrane directly to the differentiation medium. The newly added cells immediately adhere on top of the previous layers of cells and will differentiate to skeletal muscle cells.
7. On day 18, the cells and their ECM can be scarped off as a sheet using a cell scraper (Sigma-Aldrich, CLS3010-100EA).

### Example 2: Formation of Cell Sheets with Bovine Fibroblasts on Minimally Patterned Membranes Made with Isopropyl Alcohol Treated PVB Molds with Mechanical Stimulation

1. A master mold is 3D printed using Original Prusa i3 MK3S+ and PVB. Nozzle size is 0.4 mm and printing speed is 60 mm/s.
2. The mold is treated with isopropyl alcohol for 45 minutes and then left at room temperature (25° C.) over night to allow evaporation and solidification of the polymer to eliminate the patterns.
3. The membrane is made similar to the previous experiment using PDMS but treatment is performed with 1 mg/mL solution of lignin in a 1:1 mix of deionized water and methanol for 72 hours.
4. After washing, 5×10⁵ of primary bovine fibroblasts grown in their growth media (DMEM plus 10% FBS) are added to the membrane in the same media.
5. On day 3 media is refreshed with the same growth media plus enough L-ascorbate-2-phosphate to achieve a concentration of 200 μg/mL which is refreshed every two days
6. The same number of cells (5×10⁵) are added on days 4, 6, 8, and 10.
7. From days 4 to 12, mechanical stimulation is applied to the membrane by continuously stretching the membrane with an amplitude of 10% and a frequency of 1 Hz.
8. Cells and their ECM is scraped to form a sheet on day 14.

Experiment 3: Use of Decellularized ECM as Coating for Culture of Human Induced Pluripotent Stem Cells
1. Rabbit cell sheets formed in the first experiments are used here. Four of the sheets are washed with PBS to remove residues of the media. They are then submerged in 10 mL of 0.2% Triton X-100 in deionized water and are kept on ice overnight.
2. The Triton X-100 in deionized water is refreshed after 24 hours.
3. After 48 hours of treatment with decellularization solution, the sheets are spun down at 300 g for 5 minutes and are then washed with PBS three times to remove any residues of the detergent.
4. The decellularized sheets are then submerged in ice cold 4 M solution of urea in deionized water. The solution is sonicated every 12 hours using a 1 Hz frequency while on ice for two 2 second intervals.
5. After 48 hours, the ECM is completely dissolved in the solution. It is once more centrifuged at 10,000 g for 10 minutes to eliminate any floating components.
6. The solution is then dialyzed using a 1 kDa dialysis filter against deionized water for 48 hours.
7. The final solution is then frozen down at −80° C. for two hours and then freeze-dried to form the lyophilized powder.
8. The lyophilized decellularized ECM is then reconstituted in 1 mL of PBS containing 0.1 M acetic acid.
9. A non-tissue culture treated 6 well plate is then treated with 2 mL of 10×dilution of the solubilized cell derived ECM in PBS at room temperature overnight.
10. This treated well plate is then used for growing human induced pluripotent stem cells (iPSCs) (Stem Cell Technologies, 200-0511). These cells normally require coating of both tissue culture treated and non-tissue culture treated well plates with vitronectin, laminin, or Matrigel™. The Cell derived ECM solution is an alternative to these animal-derived or recombinantly produced ECM solution.

### Example 4: Formation of Cell Sheets with Bovine Myoblasts on Patterned Membranes Made with Ecoflex 00-30 Membrane on ABS Molds

9. A master mold is 3D printed using Original Prusa i3 MK3S+ and ABS. Nozzle size is 0.4 mm and printing speed is 60 mm/s.
10. The membrane is made similar to the previous experiment but using using Ecoflex 00-30 (PBAT) instead of PDMS. Components A and B of the resin are mixed with a 1:1 volume ratios and casted on the ABS mold. The curing of the resin is performed at room temperature for 4 hours. The solidified membrane is opaque with a white color that does not allow microscopy. Tannic acid treatment is performed as described herein with a 50 mg/mL solution for 72 hours.
11. After washing, 1×10⁶ of primary bovine fibroblasts grown in their growth media (DMEM plus 17% FBS, 2% horse serum, and 1% yolk extract factor) are added to the membrane in the same media.
12. On day 3 media is refreshed with the differentiation medium (DMEM plus 2% horse serum and 10 ng/mL FGF) plus 100 μg/mL L-ascorbate-2-phosphate which is refreshed every two days
13. The same number of cells (7.5×10⁵) are added on days 4, 6, and 8.
14. Cells and their ECM is scraped to form a sheet on day 18.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

### REFERENCES

The following references, to the extent that they provide exemplary procedural or other details supplementary to those set forth herein, are specifically incorporated herein by reference.
Chakrabarty, T., et al., “Bioinspired tannic acid-copper complexes as selective coating for nanofiltration membranes,” Separation and Purification Technology, 2017. 184: 188-94.
Chuah, Y. J., et al., “Simple surface engineering of polydimethylsiloxane with polydopamine for stabilized mesenchymal stem cell adhesion and multipotency,” Scientific Reports, 2015. 5(1): 18162.
Fritz, J. L. and M. J. Owen, “Hydrophobic Recovery of Plasma-Treated Polydimethylsiloxane,” The Journal of Adhesion, 1995. 54(1-4): 33-45.
Guillaume-Gentil, O., et al., “pH-controlled recovery of placenta-derived mesenchymal stem cell sheets,” Biomaterials, 2011. 32(19): 4376-84.
Li, Q., et al., “Polydopamine-collagen complex to enhance the biocompatibility of polydimethylsiloxane substrates for sustaining long-term culture of L929 fibroblasts and tendon stem cells,” Journal of Biomedical Materials Research Part A, 2018. 106(2): 408-18.
Lv, X., et al., “A one-step tannic acid coating to improve cell adhesion and proliferation on polydimethylsiloxane,” New Journal of Chemistry, 2020. 44(35): 15140-47.
Owaki, T., et al., “Cell sheet engineering for regenerative medicine: current challenges and strategies,” Biotechnol J, 2014. 9(7): 904-14.
Shahin-Shamsabadi, A. and P. R. Selvaganapathy, “rr-SACS: pH Induced Self-Assembled Cell Sheets Without the Need for Modified Surfaces,” ACS Biomater Sci Eng, 2020.6(9):5346-56.
Sharma, V., et al., “Surface characterization of plasma-treated and PEG-grafted PDMS for micro fluidic applications,” Vacuum, 2007. 81(9): 1094-1100.
Walsh, D. I., et al., “Enabling Microfluidics: from Clean Rooms to Makerspaces,” Trends in Biotechnology, 2017. 35(5): 383-92.
Wu, M.-H., “Simple poly(dimethylsiloxane) surface modification to control cell adhesion,” Surface and Interface Analysis, 2009. 41(1): 11-16.
Yamada, N., et al., “Thermo-responsive polymeric surfaces; control of attachment and detachment of cultured cells,” Die Makromolekulare Chemie, Rapid Communications, 1990. 11(11): 571-76.

What is claimed is:

1. A substrate for growing cells or a cell sheet comprising,
an elastomeric membrane having a patterned surface; and,
a hydrophilic agent attached to the surface of the elastomeric membrane.

2. The substrate of claim 1 wherein the hydrophilic agent is not an exogenous extra-cellular matrix (ECM) component.

3. The substrate of claim 1 wherein the patterned surface is molded into the elastomeric membrane.

4. The substrate of claim 1 wherein the hydrophilic agent is a polyphenol.

5. The substrate of claim 1 wherein the hydrophilic agent is selected from the group consisting of a) tannic-acid, wherein the tannic-acid is not bound to iron, and b) lignin.

6. The substrate of claim 1 wherein the elastomeric membrane comprises a silicone or PBAT.

7. The substrate claim 1 wherein the substrate has a contact angle that is at least 40 degrees and at most 85 degrees.

8. The substrate of claim 1 wherein the patterned surface comprises a portion having parallel grooves spaced apart by a distance in the range of 0.01-500 microns.

9. A method of making a substrate for growing cells or a cell sheet, the method comprising,
casting an elastomeric membrane on a mold having a patterned surface; and,
attaching a hydrophilic agent to the elastomeric membrane.

10. The method of claim 9 wherein the hydrophilic agent is not an exogenous extra-cellular matrix (ECM) component.

11. The method of claim 9 wherein the mold is made by fused deposition modeling (FDM) 3D printing.

12. The method of claim 9 wherein the treating step comprises contacting the elastomeric membrane with a solution comprising a polyphenol, wherein the solution is substantially without iron.

13. The method of claim 9 wherein the treating step comprises contacting the elastomeric membrane with a solution selected from the group consisting of a) a solution having 10-200 mg/ml of tannic acid and a pH of 7.5 or less and b) a solution having 0.1-6 mg/mL of lignin.

14. The method of claim 9 wherein the membrane comprises a silicone or PBAT.

15. A method of creating a cell construct, the method comprising:
providing an elastomeric membrane with a hydrophilic and patterned surface;
growing cells on the membrane in layers, wherein the cells produce an extra-cellular matrix (ECM); and,
removing the layers from the membrane.

16. The method of claim 15 wherein the removing step comprises scraping at least a portion of the layers from the membrane.

17. The method of claim 15 wherein the step of growing cells on the membrane in layers comprises,
adding a first set of cells to 60-90% confluence on the membrane;
incubating the first set of cells to at least 95% confluence to produce a first cell layer;
adding a second set of cells to 60-90% confluence on the first cell layer;
incubating the second set of cells to at least 95% confluence to produce a second cell layer; and,
whereby the cells of the first cell layer and the second cell layer produce an extra-cellular matrix and cells of the second cell layer attach to, or fuse together with, the cells of the first cell layer.

18. The method of any of claims 91 to 93 comprising producing the patterned surface by casting the membrane on a patterned mold.

19. The method of claim 15 wherein the elastomeric membrane comprises a silicone or PBAT.

20. The method of any of claims 91 to 95 wherein the membrane comprises a hydrophilic agent, wherein a) the hydrophilic agent is not an exogenous extra-cellular matrix (ECM) component or b) the hydrophilic agent comprises a polyphenol substantially without iron.
